(19) United States
(12) Patent Application Publication
(10) Pub. No.: US 2023/0045709 A1
(43) Pub. Date: Feb. 9, 2023

(54) PREDICTING CURVED PENETRATION PATH OF A SURGICAL DEVICE

(71) Applicant: Empress Medical, Inc., Wilmington, DE (US)

(72) Inventors: Michael Gabriel TAL, Tel Aviv (IL); Gilad MAGNAZI, Hod Hasharon (IL); Ohad HENN, Ramat gan (IL); Ron LIVNE, Haifa (IL)

(21) Appl. No.: 17/956,377

(22) Filed: Sep. 29, 2022

Related U.S. Application Data
(63) Continuation of application No. PCT/US2021/024270, filed on Mar. 26, 2021.
(60) Provisional application No. 63/001,808, filed on Mar. 30, 2020.

(51) Int. Cl. A61B 34/10; A61B 17/04; A61B 90/00

(52) U.S. Cl. CPC A61B 34/10; A61B 17/0482; A61B 90/39; A61B 2090/3937; A61B 2034/107; A61B 2090/373; A61B 2090/306

(57) ABSTRACT

A surgical device comprising an elongated body, a tissue penetrating apparatus and a light projector. The elongated body can reach with distal end thereof a surface of an organ within a subject's body. The tissue penetrating apparatus can be extended from the elongated body distal end along a curved penetration path restricted to a chosen penetration plane. The light projector can generate a shaped illumination on the surface of the organ indicative of an intersection of the penetration plane with the surface of the organ.

### CROSS-REFERENCE TO RELATED APPLICATIONS

This application is a continuation of PCT/US2021/024270 filed on Mar. 26, 2021, and further claims the benefit under 35 U.S.C. § 119 of U.S. Provisional Patent Application No. 63/001,808, filed on Mar. 30, 2020, both of which are entitled PREDICTING CURVED PENETRATION PATH OF A SURGICAL DEVICE, and the entire contents of both of which are hereby incorporated by reference herein.

### FIELD OF THE INVENTION

The present disclosure relates to devices and methods for planning a surgical path in a body of a subject, and more particularly, but not exclusively, to devices and methods for estimating and/or predicting location and/or orientation of a curved penetration path of a surgical device (e.g., device comprising a curving needle), such as for avoiding harm to adjacent organ or other tissues with the surgical device.

### BACKGROUND OF THE INVENTION

Some operations in minimally invasive surgery require penetration of an internal organ in a non-straight or curved path. For example, suturing an organ includes passing a needle across tissues forming the organ, forward and then backward, from an entry point to an exit point at the organ surface located at same side thereof, relative to the minimally invasive entry port. Minimally invasive surgery is usually performed under vision using an endoscope that protrudes into the body towards the treated area via a dedicated port.

One of the challenges in minimally invasive surgeries requiring curved penetration paths by a surgical device, especially ones that include large-curvature needles, is to avoid harm to other tissues or organs not related to the procedure. For example, the uterus is located adjacent to the urine bladder, so operations involving puncturing of the uterus (such as in process of suturing tissues thereof) may potentially cause unintentional puncturing of the bladder.

Therefore, there is a need to provide surgeons with devices and methods for estimating and/or predicting one or more curved penetration paths, and/or particular points along such paths, of a surgical device through organs or other tissues in the body.

### SUMMARY OF THE INVENTION

The present disclosure relates to devices and methods for planning a surgical path in a body of a subject, and more particularly, but not exclusively, to devices and methods for estimating and/or predicting location and/or orientation of a curved penetration path of a surgical device (e.g., device comprising a curving needle), such as for avoiding harm to adjacent organ or other tissues with the surgical device.

In certain embodiments, there is provided a surgical device which can comprise: (a) an elongated body comprising a longitudinal axis, configured to pass through an opening in a subject's body and to reach, with a distal end thereof, a surface of an organ within the subject's body; (b) a tissue penetrating apparatus configured to extend from the elongated body distal end through tissue layers of the organ, along a curved penetration path restricted to a chosen penetration plane (“plane” refers herein to a two-dimensional flat surface); and (c) a light projector connected to the elongated body proximally to the elongated body distal end, configured to generate a shaped illumination projectable on the surface of the organ indicative of an intersection of the penetration plane with the surface of the organ.

In some embodiments, the surgical device is configured such that the shaped illumination denotes location and orientation of the intersection of the penetration plane with the surface of the organ.

In some embodiments, the surgical device is configured such that the shaped illumination denotes one or more portions of an intersection line representing the intersection of the penetration plane with the surface of the organ.

In some embodiments, the surgical device is configured such that the light projector projects at least one beam restricted to travel substantially along the penetration plane.

In some embodiments, the at least one beam is a line-shaped beam having a substantially line-shaped cross-section configured to substantially coincide with the penetration plane.

In some embodiments, the light projector is configured to project the at least one beam in a generally distal direction at an angle to the longitudinal axis.

In some embodiments, the surgical device is configured such that the shaped illumination is formed of a series of spots being spaced apart and/or partially merged with each other.

In some embodiments, the spots are arranged substantially inline relative to the longitudinal axis.

In some embodiments, most or all the spots are arranged along a cross-sectional plane comprising the longitudinal axis.

In some embodiments, the cross-sectional plane is the penetration plane.

In some embodiments, each spot is a footprint of a separate ray of a line-shaped beam, each ray forms a different projection angle with the longitudinal axis.

In some embodiments, the light projector includes a laser source.

In some embodiments, the laser source includes an optical fiber such as a single-mode optical fiber.

In some embodiments, the laser source is located within a hollow projector body comprising an opening or optical window located at a lateral wall potion thereof configured to transmit a line-shaped beam in a chosen fan angle.

In some embodiments, the fan angle is at an angle to the longitudinal axis.

In some embodiments, the light projector further comprising a collimating lens, configured to generate a collimated beam from a pre-collimated laser beam projected from the laser source, and further comprising a beam-line lens configured to generate the line-shaped beam from the collimated beam.

In some embodiments, the light projector further comprising a reflective surface tilted relative to the longitudinal axis, configured to reflect and direct the line-shaped beam in the chosen fan angle, along the penetration plane, through the opening or optical window of the projector body.

In some embodiments, the distal end of the elongated body comprises a sharp tip configured for forming an entry opening on the surface of the organ when penetrating the organ.

In some embodiments, the tissue penetrating apparatus includes a curved needle configured to pass straighten along the longitudinal axis in a first lumen enclosed by the longitudinal body, and to voluntarily deform to a less-elastically stressed curved shape when a needle emerging portion thereof emerges from the first lumen distally to the distal end of the elongated body.

In some embodiments, the surgical device is configured such that the curved needle forms the curved penetration path when the needle emerging portion is advanced within the organ parallelly to the penetration plane.

In some embodiments, the tissue penetrating apparatus further comprising a stylet configured to pass through a second lumen enclosed by the curved needle until a chosen length of a stylet emerging portion thereof emerges from the second lumen distally to a distal end of the curved needle.

In some embodiments, the surgical device further comprising visible markers on elongated body indicative of a spatial orientation of the curved penetration path over the penetration plane, relative to a visual line of sight directed generally towards the visible markers.

In some embodiments, the visible markers include a distal circular marker and a proximal circular marker surrounding the cylindrical portion perpendicularly to the longitudinal axis.

In some embodiments, the visible markers include a discrete marker.

In some embodiments, the discrete marker is provided between the distal circular marker and a proximal circular marker.

In certain embodiments, a system is provided which comprises at least one processor for processing a digital image capturing a portion of the elongated body in the subject's body relative to the line of sight. In some embodiments, the at least one processor is configured to: locate the visible markers and at least one contour line of the elongated body, calculate relative positions and/or distances between the visible markers and the at least one contour line, and extrapolate the spatial orientation.

In some embodiments, the at least one processor is configured to produce a penetration path graph for predicting the penetration path location and orientation in the organ based on the extrapolated spatial orientation and predetermined shape and size values of the tissue penetration apparatus when fully extended from the distal end of the elongated body.

In some embodiments, the system includes or is connectable to a screen and configured to illustrate a graphic representation of the penetration path graph over the digital image on the screen.

In certain embodiments there is a surgical device, which can comprise: (a) an elongated body comprising a longitudinal axis, configured to pass through an opening in a subject's body and to reach, with a distal end thereof, a surface of an organ within the subject's body; (b) a tissue penetrating apparatus configured to extend from the elongated body distal end through tissue layers of the organ, along a curved penetration path restricted to a chosen penetration plane; and (c) a light projector connected to the elongated body proximally to the elongated body distal end, configured to project a laser line-shaped beam having a line-shaped cross-section substantially coinciding with the penetration plane.

In some embodiments, the light projector is configured to project the line-shaped beam in a generally distal direction at an angle to the longitudinal axis.

In some embodiments, the light projector is configured to project the line-shaped beam is in a chosen fan angle.

In some embodiments, the fan angle is at an angle to the longitudinal axis.

In certain embodiments there is provided a surgical device, which can comprise: (a) an elongated body configured to pass through an opening into an inner volume in a subject's body and to reach, with a distal end thereof, a surface of an organ within the subject's body located generally in front of the opening; (b) a tissue penetrating apparatus configured to extend via the elongated body distal end through tissue layers of the organ, along a curved penetration path, from an entry point to an exit point spaced apart from the entry point at the surface of the organ; and (c) a light projector connected to the elongated body proximally to the elongated body distal end, configured to project at least one beam for generating a shaped illumination on the surface of the organ indicative of a predicted location of the exit point.

In some embodiments, the curved penetration path is restricted to a chosen penetration plane and comprising the entry point and the exit point.

In some embodiments, the shaped illumination is indicative of an intersection of the penetration plane with the surface of the organ.

In some embodiments, the at least one beam includes a laser line-shaped beam having a line-shaped cross-section substantially coinciding with the penetration plane.

In some embodiments, the light projector is configured to project the at least one beam in a generally distal direction at an angle to the longitudinal axis.

In certain embodiments, there is provided a surgical device, which can comprise: (a) an elongated body having a cylindrical portion and a centerline coinciding with a longitudinal axis; (b) a tissue penetrating apparatus configured for extending from the elongated body distal end along a curved penetration path restricted to a chosen penetration plane; and (c) visible markers on the cylindrical portion of the elongated body indicative of a spatial orientation of the curved penetration path over the penetration plane, relative to a visual line of sight directed generally towards the visible markers.

In some embodiments, the visible markers include a distal circular marker and a proximal circular marker surrounding the cylindrical portion perpendicularly to the longitudinal axis.

In some embodiments, the visible markers include a discrete marker.

In some embodiments, the discrete marker is provided between the distal circular marker and a proximal circular marker.

In certain embodiments, there is provided a system comprising at least one processor for processing a digital image capturing a portion of the elongated body the subject's body relative to the line of sight. In some embodiments, the at least one processor is configured to: locate corners formed by intersections of the distal circular marker, the proximal circular market and at least one contour line of the elongated body, for determining orientation of the longitudinal axis; calculate relative positions and/or distances between the discrete marker and the corners, for determining orientation of the penetration plane relative to the longitudinal axis; and extrapolate the spatial orientation of the curved penetration path over the penetration plane.

In some embodiments, the at least one processor is configured to produce a penetration path graph for predicting the penetration path location and orientation in the organ based on the extrapolated spatial orientation and predetermined shape and size values of the tissue penetration apparatus when fully extended from the distal end of the elongated body.

In some embodiments, the system includes or is connectable to a screen and configured to illustrate a graphic representation of the penetration path graph over the digital image on the screen.

In certain embodiments, there is provided a method which can comprise at least one of the followings (not necessarily in same order): positioning a surgical device according to claim 35 in a subject's body such that the distal end of the elongated body engages with a surface of the organ; recording a digital image capturing the surface of the organ and the visible markers from the visual line of sight; processing the image to determine the spatial orientation of the curved penetration path over the penetration plane, relative to the visual line of sight; producing a penetration path graph of a predicted penetration path location and orientation in the organ based on the extrapolated spatial orientation and predetermined shape and size values of the tissue penetration apparatus when fully extended from the distal end of the elongated body; and illustrating on a screen a graphic representation of the penetration path graph over the digital image.

In some embodiments, the visible markers include and a distal circular marker and a proximal circular marker surrounding the cylindrical portion perpendicularly to the longitudinal axis, and a discrete marker provided in proximity to and/or between the distal circular marker and a proximal circular marker. In some such embodiments, the processing includes: locating corners formed by intersections of the distal circular marker, the proximal circular market and at least one contour line of the elongated body, for determining orientation of the longitudinal axis; calculating relative positions and/or distances between the discrete marker and the corners, for determining orientation of the penetration plane relative to the longitudinal axis; and extrapolating the spatial orientation of the curved penetration path over the penetration plane.

In some embodiments, the method further comprising predicting an exit point of the tissue penetrating apparatus from the organ by identifying an intersection of the graphic representation with a shaped illumination on the surface of the organ, shown in the digital image, indicative of an intersection of the penetration plane with the surface of the organ.

In some embodiments, the surgical device further comprising a light projector connected to the elongated body proximally to the elongated body distal end, configured for projecting a laser line-shaped beam having a line-shaped cross-section substantially coinciding with the penetration plane. In some such embodiments, the method include projecting the laser line-shaped beam to generate the shaped illumination on the surface of the organ.

In certain embodiments, there is provided a method which can comprise at least one of the followings (not necessarily in same order): providing a surgical device comprising an elongated body, a tissue penetrating apparatus configured to extend from a distal end of the elongated body along a curved penetration path, and a light projector positioned proximally to the elongated body distal end; engaging the elongated body distal end with a surface of a target organ in a subject's body; generating a shaped illumination on the surface of the target organ using the light projector; based on position and/or orientation of the shaped illumination relative to a non-target tissue located adjacent to the target organ, choosing a penetration plane for the curved penetration path so as to avoid passing the tissue penetrating apparatus through the non-target tissue; and advancing the tissue penetrating apparatus in the target organ along the curved penetrating path restricted to the chosen penetration plane.

In some embodiments, the method comprising fixating the elongated body distal end to the surface of the target organ so as to resist rotation of the shaped illumination on the surface of the organ relative to a longitudinal axis of the elongated body.

In some embodiments, the generating includes projecting at least one beam restricted to travel substantially along the penetration plane towards the surface of the organ in a generally distal direction at an angle to the longitudinal axis.

In some embodiments, the method comprising passing an implantable member or a suture along the curved penetration path.

All technical or/and scientific words, terms, or/and phrases, used herein have the same or similar meaning as commonly understood by one of ordinary skill in the art to which the invention pertains, unless otherwise specifically defined or stated herein. Illustrative embodiments of methods (steps, procedures), apparatuses (devices, systems, components thereof), equipment, and materials, illustratively described herein are exemplary and illustrative only and are not intended to be necessarily limiting. Although methods, apparatuses, equipment, and materials, equivalent or similar to those described herein can be used in practicing or/and testing embodiments of the invention, exemplary methods, apparatuses, equipment, and materials, are illustratively described below. In case of conflict, the patent specification, including definitions, will control.

### BRIEF DESCRIPTION OF THE DRAWINGS

Some embodiments are herein described, by way of example only, with reference to the accompanying drawings. With specific reference now to the drawings in detail, it is stressed that the particulars shown are by way of example and for purposes of illustrative description of some embodiments. In this regard, the description taken together with the accompanying drawings make apparent to those skilled in the art how some embodiments may be practiced.

In the drawings:

FIG. 1 shows a block diagram comprising an exemplary system for predicting curved penetration path of a surgical device in an organ in a body of a subject, according to some embodiments;

FIGS. 2A-2B schematically illustrate respectively isometric view and cross-sectional view of an exemplary variation of the system of FIG. 1 shown in use for treating an organ, according to some embodiments;

FIGS. 3A-3F schematically illustrate exemplary scenarios representing steps in an exemplary method for treating an organ in the body using the system of FIG. 1, according to some embodiments;

FIGS. 4A-4C illustrate views of an exemplary surgical device comprising a tissue penetrating apparatus and a light projector, according to some embodiments;

FIG. 4D illustrates an isometric view of an exemplary light projector provided with the surgical device shown in FIG. 4A, according to some embodiments;

FIG. 4E illustrate an exemplary variation of the surgical device shown in FIG. 4A, according to some embodiments;

FIGS. 5A-5C schematically illustrate exemplary components of the light projector shown in FIG. 4C, according to some embodiments;

FIGS. 6A-6B illustrate portions of the surgical device of FIG. 4A comprising exemplary visible markers, according to some embodiments; and

FIG. 7 illustrate an exemplary graph representing possible relationship between variables related to a captured image of the visible markers of the surgical device, according to some embodiments.

### DETAILED DESCRIPTION

Certain embodiments relate to systems, devices and methods for planning a surgical path in a body of a subject, and more particularly, but not exclusively, to systems, devices and methods for estimating and/or predicting location and/or orientation of a curved penetration path of a surgical device (e.g., device comprising a curving needle), such as for avoiding harm to adjacent organs or other tissues with the surgical device.

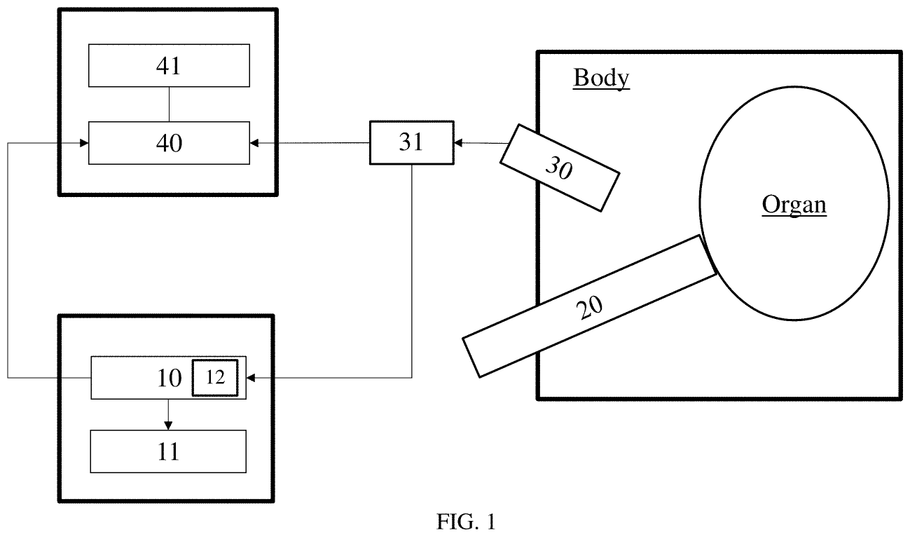

FIG. 1 shows a block diagram comprising an exemplary system 10 put in use for predicting curved penetration path of a surgical device 20 in an organ in a body of a subject. System 10 is configured for capturing and/or analyzing an image of the surgical device within the body, for assessing orientation of the surgical device and/or for predicting the curved penetration path in the organ. The system may include at least one processor 12 for processing a digital image capturing a portion of the elongated body in the subject's body relative to the line of sight. The processor(s) 12 can be configured (e.g., programmed) to locate visible markers and/or at least one contour line of the elongated body, calculate relative positions and/or distances between the visible markers and the at least one contour line, and extrapolate the spatial orientation of the curved penetration path.

System 10 includes, or is operatively connectable to, a system screen 11 for displaying images and/or data for facilitating communication between system 10 and a user. System 10 may include or be operatively connectable with an intra-body image capturing device, such as an endoscope 30, optionally in a form of a laparoscope, configured for protrusion into subject's body via a dedicated laparoscopic port. Modern operating rooms equipped for minimally invasive surgeries will normally include image viewing equipment connectable to endoscope 30, such as a laparoscopic tower 40 which can produce, display or project images such as via a laparoscopy screen 41. In order to receive images (optionally concurrently) with laparoscopic tower 40, system 10 may be connected to endoscope 30 via a signal splitter 31. In order to add visual representation of a predicted curved penetration path, points thereon, margin of error, and/or other information, over the original image captured by endoscope 30, system 10 can be connected also to laparoscopic tower 40 and allowed to process or edit captured and/or processed images, and/or superimpose visuals over images shown in laparoscopy screen 41.

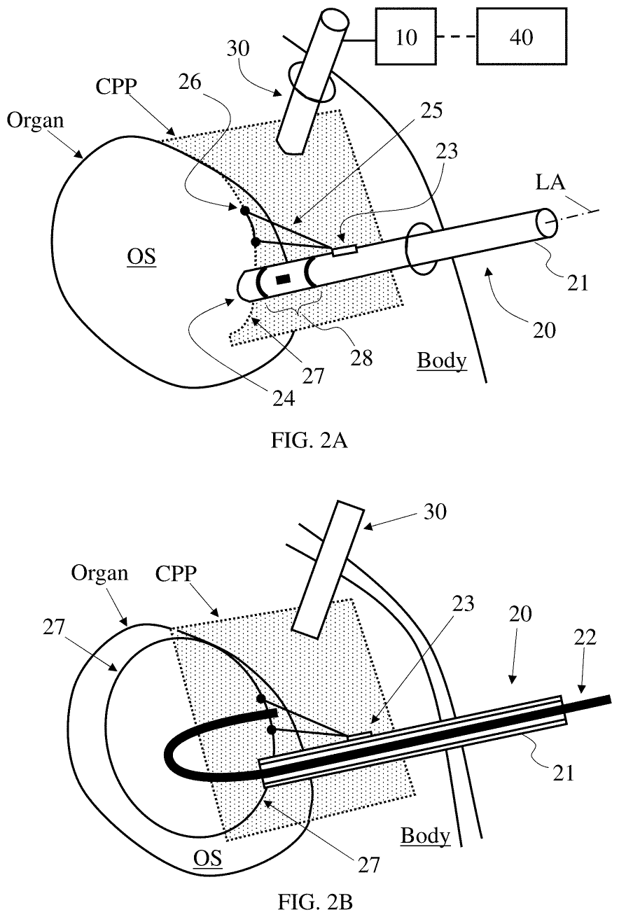

FIGS. 2A-2B schematically illustrate respectively isometric view and cross-sectional view of an exemplary implementation of system 10 shown in use for treating an organ within the subject's body. Surgical device 20 includes an elongated body 21, a tissue penetrating apparatus 22 extendable via elongated body 21, and a light projector 23. Elongated body 21 is configured for passing through an opening in a subject's body and for engaging, with a distal end 24 thereof, a surface OS of the organ. The tissue penetrating apparatus 22 is configured for extending from elongated body distal end 24 through tissue mass forming the organ, along a curved penetration path restricted to a chosen penetration plane CPP (i.e., a two-dimensional flat surface). The chosen penetration plane CPP is fixed relative to a longitudinal axis LA which coincides with a centerline of elongated body 21.

Light projector 23 is connected to elongated body 21, proximally to the elongated body distal end 24, and is selectively operable for projecting at least one beam 25 restricted to travel substantially along penetration plane CPP. As such, light projector 23 is configured to generate (e.g., projecting) a shaped illumination 26 on organ surface OS that is indicative of an intersection 27 of penetration plane CPP with organ surface OS when elongated body distal end 24 engages the organ surface OS.

Surgical device 20 may also include visible markers 28 on one or more portions of the elongated body 21, which can be indicative of a spatial orientation of the curved penetration path over the penetration plane CPP, relative to visual line of sight of endoscope 30 directed generally towards the visible markers 28.

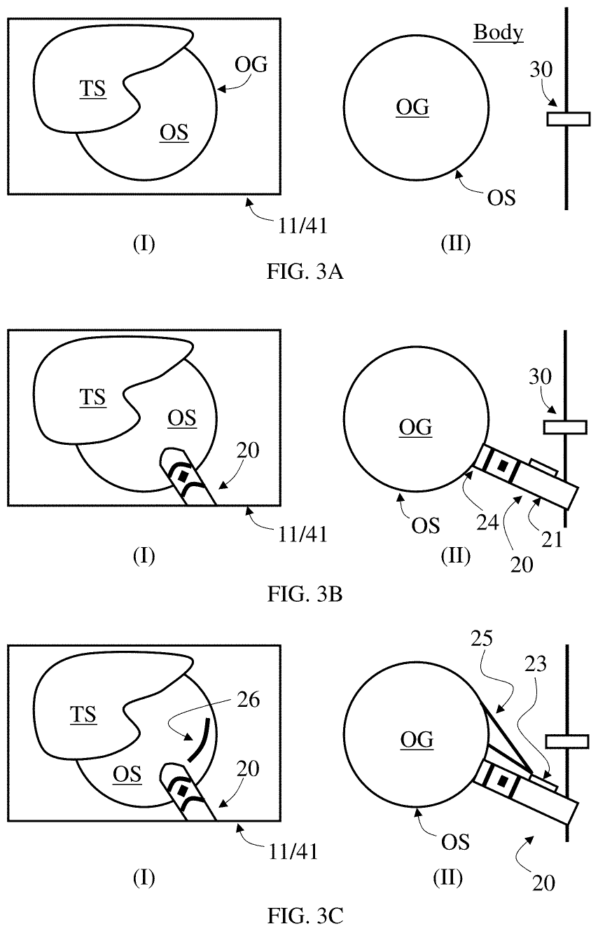

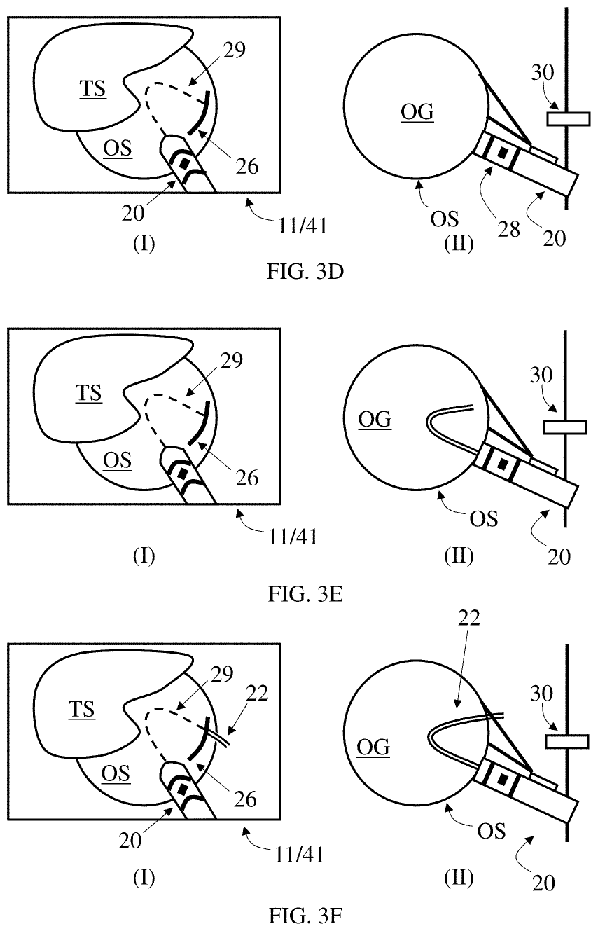

FIGS. 3A-3F schematically illustrate exemplary scenarios representing steps in an exemplary method for treating an organ OG in the body using system 10, in accordance with the deployment scheme shown in FIG. 2. Each figure shows a separate scenario in two concurrent views: a first view (I) which is a schematic isometric illustration of the images shown in system screen 11 and/or in laparoscopy screen 41 during the surgical procedure with the visuals created and superimposed by system 10 over the original image captured by endoscope 30; and a second view (II) which is a schematic cross-sectional illustration of the surgical procedure inside the subject's body. For demonstrative purposes, the cross section in second view (II) coincides with penetration plane CPP in accordance with surgical device 20 orientation shown in the figures.

FIG. 3A shows a first scenario in which endoscope 30 is protruding into the subject's body and can visualize the target treatment area of organ OG, as well as adjacent tissue TS that puncturing or harming thereof is to be avoided or minimized during piercing and progressing in organ OG with surgical device 20. FIG. 3B shows a second scenario in which surgical device 20 is introduced into the subject's body with distal portion of elongated body 21 protruding into a body cavity (e.g., the abdominal cavity) via an opening or laparoscopic port. Surgical device 20 is advanced distally and/or maneuvered until elongated body distal end 24 engages (e.g., touches, pressed against and/or fixated to) organ outer surface OS of organ OG.

FIG. 3C shows a third scenario in which light projector 23 is operated and applied to projected one or more beams 25 that form on organ surface OS at least one illumination 26 (e.g., resembling a line) that is visually and/or geometrically indicative of the intersection of penetration plane CPP with organ surface OS. As shown in FIG. 3D, system 10 can also be applied to analyze relative positioning of visible markers 28, and/or portions or interactions thereof, for calculating an estimated or predicted curved penetration path of tissue penetrating apparatus 22 in organ OG, and for optionally superimposing a graphic representation 29 of the predicted curved penetration path over the original endoscope image, as appeared on the screen (system screen 11 and/or in laparoscopy screen 41).

The intersection of illumination 26 with graphic representation 29, as appears on the screen, can be used for calculating, estimating or predicting an exit point formable by tissue penetrating apparatus 22, if applied to penetrate into organ OG to an extent sufficient for protruding backwards (e.g., generally towards endoscope 30) across organ surface OS via the predicted exit point. Surgical device 20 can be maneuvered (e g, manually, automatically or robotically) relative to organ surface OS for reaching a chosen spatial orientation of surgical device 20 for determining location and/or orientation of illumination 26 on organ surface OS and of curved penetration path in organ OG. The spatial orientation can be chosen so as to avoid unnecessary potential harm to adjacent tissue TS when tissue penetrating apparatus 22 is applied. In some embodiments, surgical device 20 is used for surrounding (e.g., encircling) a target tissue mass within organ OG with tissue penetrating apparatus 20, and the spatial orientation can be chosen so as to arrive to a preferred surrounding path, possibly one out of a number of differently oriented curved paths. Such a target tissue mass can include a portion of a tumor, and its surrounding can be applied for passing a tension member around it, at least partially, as described in U.S. patent application Ser. No. 16/539,800, for example.

FIG. 3E shows a fifth scenario in which tissue penetrating apparatus 22 is applied to penetrate organ OG and advance therein, optionally substantially along the predetermined chosen curved penetration path. FIG. 3F shows an optional scenario that can take place if tissue penetrating apparatus 22 is used for crossing through organ surface OS from the inside out, optionally substantially through or adjacent to the predicted exit point. Advancing of tissue penetrating apparatus 22 through organ OG and/or surface OS may or may not be performed under noninvasive imaging (e.g., by way of radiography or ultrasound).

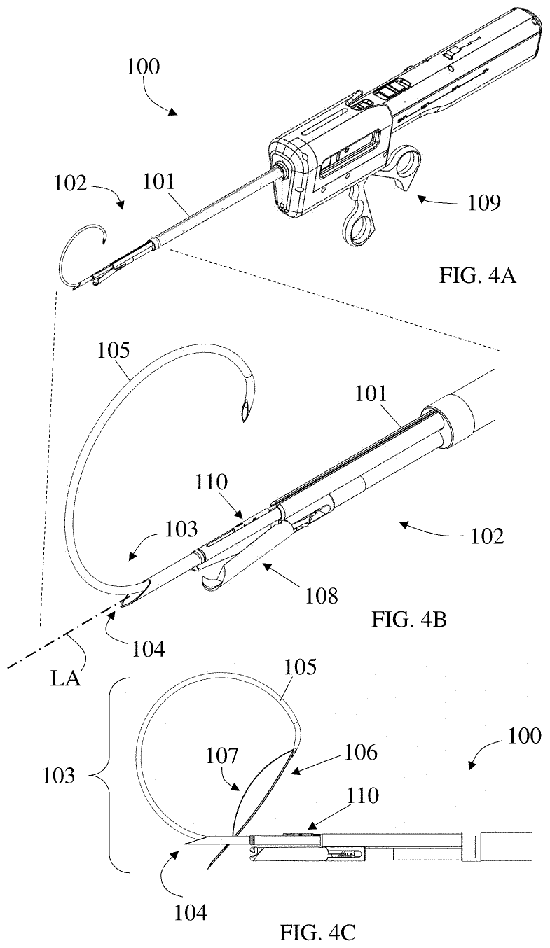

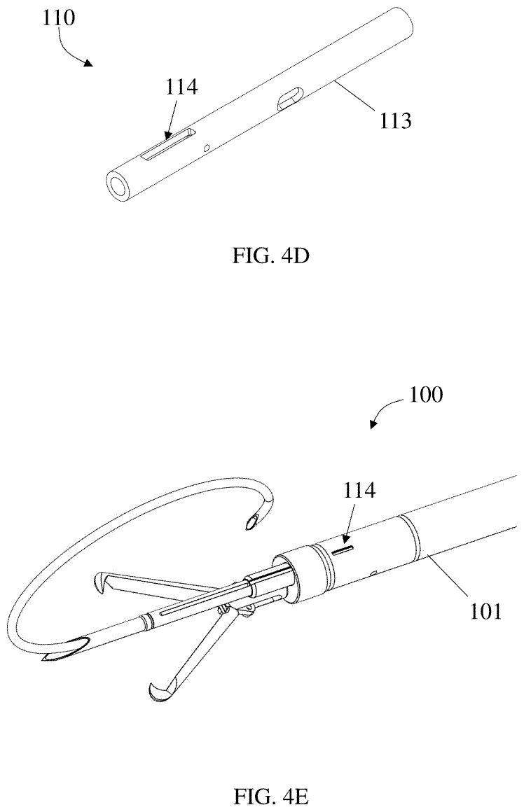

FIGS. 4A-4C illustrate an exemplary surgical device 100, which is optionally similar or identical in at least one structural and/or functional feature to surgical device 20; and, as such, may be configured for deployment such as in the exemplary deployment shown in FIGS. 2 and/or applied in an exemplary method that can include one or more of the scenarios shown in FIG. 3. FIG. 4A illustrates a full isometric view of surgical device 100 and FIG. 4B illustrates a partial magnified view of surgical device 100 distal potion. Surgical device 100 includes an elongated body 101 having a longitudinal axis LA coinciding with centerline thereof. Elongated body 101 is configured for passing through an opening in a subject's body and engaging, with a distal end 102 thereof, a surface of an organ within the subject's body (similarly to as shown in FIGS. 2, for example).

Surgical device 100 also includes a tissue penetrating apparatus 103 configured for extending from elongated body distal end 102 through tissue mass forming the organ, along a curved penetration path restricted to a chosen penetration plane CPP (shown in FIG. 7, for example). Penetration plane CPP includes longitudinal axis LA and is fixed relative thereto, such that axis LA coincides with and extends along the penetration plane CPP so by repositioning surgical device 100 relative to the organ, each one of longitudinal axis LA, penetration plane CPP and the curved penetration path, will change accordingly while remaining fixedly positioned relative to each other.

Surgical device 100 includes a sharp tip 104 configured for forming an entry opening on the surface of the organ for penetrating into the organ. Sharp tip 104 may be fixed to distal end 102 of elongated body 101 or provided at a distal end of a member of tissue penetrating apparatus 103, which can be in a form of a needle or cannula slidable relative to elongated body distal end 102. Tissue penetrating apparatus 103 includes a curved needle 105 slidable relative to sharp tip 104. Curved needle 105 is configured for passing straighten along longitudinal axis LA in a first lumen enclosed by elongated body 101, and to voluntarily deform to a less-elastically stressed curved shape when a portion thereof emerges from first lumen 106 distally to elongated body distal end 102. This allows surgical device 100 to form a curved penetration path with curved needle 105 by advancing its re-curved emerging portion within the organ parallel to the penetration plane.

As shown in FIG. 4C, a stylet 106 can be included as part of tissue penetrating apparatus 103, and can be configured for passing through a second lumen enclosed by curved needle 105 such that a chosen length thereof (stylet emerging portion) emerges from the second lumen distally to a distal end of the curved needle 105. Stylet 106 can be a flexible rod-like member optionally having a sharp tip configured for puncturing through the organ tissue. Stylet 106 may be equipped with capturing means such as for facilitating selective physical capturing of a wire (e.g., suture wire) and/or tissue; such capturing means may include a wire member 107 extending along between two connections points on a distal portion of stylet 106. After passing a suture between the wire member 107 and stylet 106, the suture can be secured to tissue penetrating apparatus 103 by pulling it with stylet 106 and keeping it secured continuously against distal end of curved needle 105. Once secured, the suture can be pulled or pushed with tissue penetrating apparatus 103 along the curved penetration path in the organ.

Surgical device 100 may include a tissue anchoring mechanism, which can comprise as a grasper or tenaculum 108, for holding a portion of the organ when penetrating the organ and/or advancing through its tissues with tissue penetrating apparatus. Surgical device 100 may include at least one actuator 109 provided at a proximal portion thereof, configured for facilitating selective applicability (e.g., advancing or withdrawing with sufficient force) of at least one of the tissue penetrating apparatus 103, the sharp tip 104, the curved needle 105, the stylet 106, and the grasper or tenaculum 108.

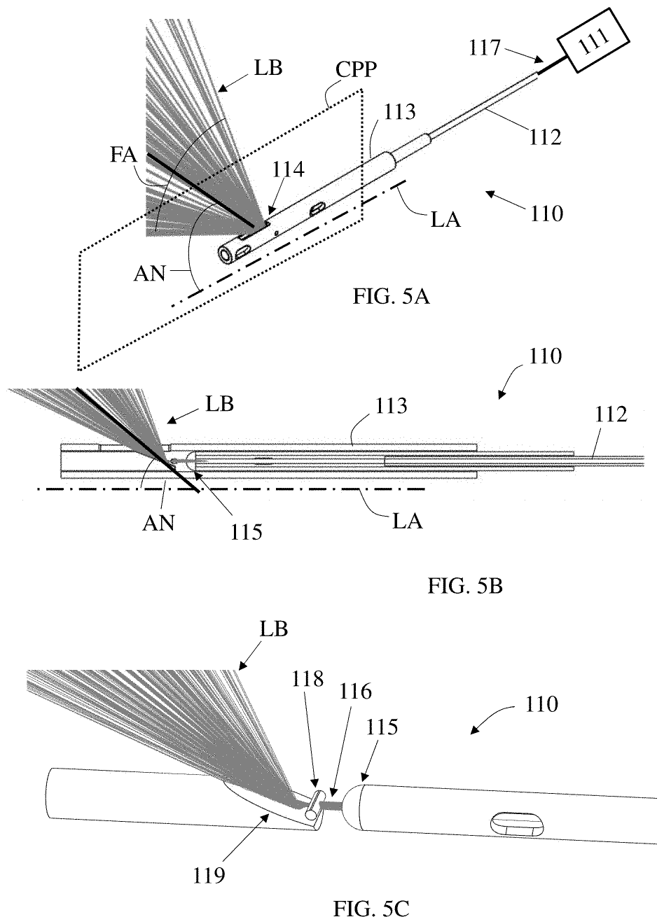

The surgical device 100 also includes a light projector 110 connected to elongated body 101 proximally to its distal end 102. FIG. 4D illustrates light projector 110 as a standalone component, although it is normally provided fixated to outer boundary of elongated body 101, as shown in FIG. 4B. FIG. 4E shows an alternative embodiment, although similar in some or all other structural and/or functional features, in which light projector 110 or a similar apparatus (not shown) is located inside elongated body 101 and configured to project light via an opening or optical window 114 merging with wall of elongated body 101. FIGS. 5A-5C schematically illustrate exemplary components of light projector 110, when in use. Light projector 110 is selectively operable for projecting at least one line-shaped beam LB, optionally comprising out of a plurality of rays, having a substantially line-shaped cross-section configured for substantially coinciding with the penetration plane. Beam LB is restricted to travel substantially along the penetration plane CPP in a generally distal direction at an angle to the longitudinal axis LA.

Similarly to as previously described with respect to FIGS. 2 and 3, when elongated body distal end 102 engages the organ surface, and optionally fixed thereto, beam LB projected by light projector 110 generates a shaped illumination on the surface of the organ indicative of an intersection of the penetration plane with the surface of the organ. The shaped illumination may be in a form of a line and/or as a series of spots spaced apart and/or partially merged with each other, arranged substantially inline relative to longitudinal axis LA. Each spot can be a footprint of a separate ray of line-shaped beam LB, each ray forms a different projection angle with longitudinal axis LA, and most or all the spots are arranged along the penetration plane. The shaped illumination denotes one or more portions of an intersection line representing the intersection of the penetration plane with the surface of the organ which is indicative of the location and orientation of the intersection of the penetration plane with the surface of the organ.

Light projector 110 includes a laser source 111 comprising or coupled with an optical fiber 112, optionally a single-mode optical fiber. Light projector 110 includes a hollow projector body 113 comprising an opening or optical window 114, located at a lateral wall potion of projector body 113, that is configured to transmit a line-shaped beam in a chosen fan angle FA, which is the angular spread of a laser beam that can determine the size of a projection (such as a line or cross) at a particular distance. Fan angle FA is at an angle AN (e.g., with a median thereof) to the longitudinal axis LA. Light projector 110 includes a collimating lens 115, configured for generating a collimated beam 116 from a pre-collimated laser beam 117 projected from laser source 111, and also a beam-line lens 118 configured for generating the line-shaped beam LB from collimated beam 116. A light reflecting surface (e.g., mirror) 119 is optionally provided in projector body 113 and tilted relative to longitudinal axis LA, and is configured to reflect and direct line-shaped beam LB in the chosen fan angle FA, along the penetration plane, through the opening or optical window 114.

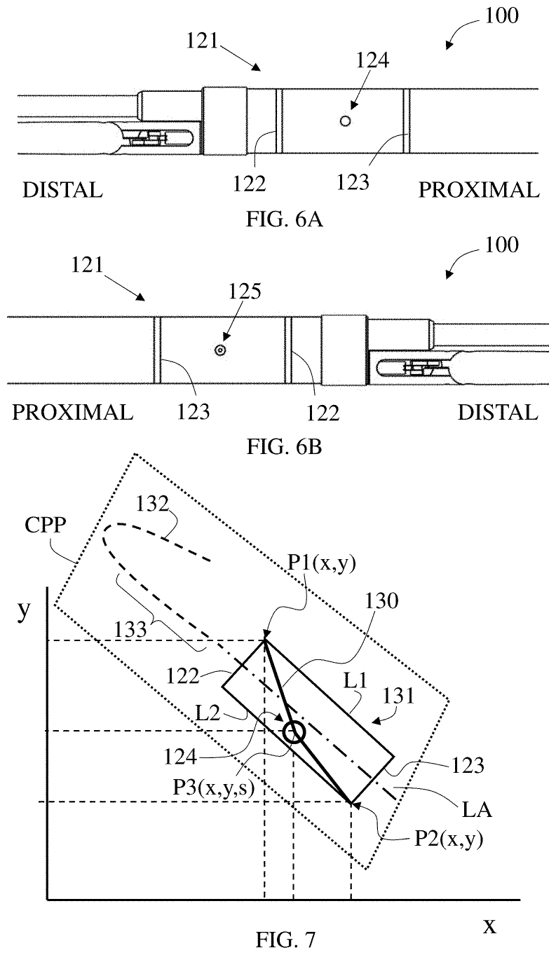

Surgical device 100 includes visible markers on elongated body 101 indicative of a spatial orientation of the curved penetration path over the penetration plane, relative to a visual line of sight directed generally towards the visible markers. FIGS. 6A-6B illustrate a cylindrical portion 121 of elongated body 101 comprising the visible markers. The visible markers include a distal circular marker 122 and a proximal circular marker 123, each one surrounds (encircles) cylindrical portion 121 perpendicularly to longitudinal axis LA. The visible markers also include two diametrically opposing discrete markers—a first discrete marker 124 and a second discrete marker 125—which are visibly distinct from each other by at least shape and/or size and are both provided between distal circular marker 122 and proximal circular marker 123 at opposing sides of the cylindrical portion 121. Optionally and as shown, first discrete marker 124 is configured as a plain circle and second discrete marker 125 is configured as two concentric circles.

In some embodiments, system 10 is configured to analyze intra-body captured images of the visible markers of surgical device 100 (similarly to as described in relation to visible markers of surgical device 20). Reference is made to FIG. 7, which illustrates an exemplary graph 130 representing possible relationship between variables related to an exemplary captured image (similar to the image sown in first view (I) in FIG. 3C, for example) of the visible markers of surgical device 100. System 10 may plot such a graph, or it may calculate or produce series of numbers significantly correlated to an equation derivable from or resembling graph 130. Referring back to FIG. 1, system 10 includes at least one processor 12 configured for processing a digital image that captures (relative to a chosen line of sight) a portion of elongated body 101, comprising at least cylindrical portion 121, in the subject's body.

In some embodiments, processor 12 is configured (e.g., programmed to apply or exercise a dedicated algorithm) to locate (e.g., detect or assess location of) at least two corners P1 and P2 of a rectangle 131 formed by the visual representations of distal circular marker 122, proximal circular marker 123, a first contour line L1 of elongated body 101 (extending between distal circular marker 122 and proximal circular marker 123 along cylindrical portion 121), and a second contour line L2 of elongated body 101 (extending between distal circular marker 122 and proximal circular marker 123 opposingly to first contour line L1). First corner P1 is formed by intersection of distal circular marker 122 with first contour line L1, and second corner P2 is formed by intersection of proximal circular marker 123 with second contour line L2. Optionally and as shown, first corner P1 and second corner P2 denote opposing ends of a diagonal to rectangle 131. Processor 12 is also configured to locate (e.g., detect or assess location of) an intermediate point P3 according to position of the discrete marker shown in the captured image. Optionally and as shown, intermediate point P3 denotes center of first discrete marker 124 as visually represented in the captured image.

In some embodiments, processor 12 is configured to determine orientation of longitudinal axis LA in the captured image by calculating relative positions and/or distances between corners P1 and P2. Processor 12 is optionally configured to determine orientation of penetration plane CPP, optionally relative to (e.g., rotated about) longitudinal axis LA, by calculating relative positions and/or distances of corners P1 and P2, and intermediate point P3. In some embodiments, processor 12 is configured to extrapolate the spatial orientation of a predetermined penetration path graph 132 over penetration plane CPP. The shape and size of penetration path graph 132 can be calculated or retrieved from memory of system 10 in correlation with measured shape and size of a predetermined protruding length of curved needle 105 between curve needle distal tip and sharp tip 104 provided in a substantially relaxed (non-stressed) state.

Processor 12 can then extrapolate spatial orientation of an initial portion 133 of penetration path graph 132 since that entire length of path graph 132 is a priori in coincidence with already determined penetration plane CPP and initial portion 133 thereof is substantially straight and extends from sharp tip 104 parallel to longitudinal axis LA. Orientation of penetration path graph 132 following its initial portion 133, if it curves generally towards first corner P1 or if in an opposite direction, can be determined by processor 12 by linking intermediate point P3 to either one of first discrete marker 124 or second discrete marker 125. For example, in case system 10 links intermediate point P3 with first discrete marker 124 (as shown in FIG. 7, for example) processor 12 is configured to determine that penetration path graph 132 curves generally towards first corner P1, whereas if system 10 links intermediate point P3 with second discrete marker 125 processor is configured to determine that penetration path graph 132 curves generally towards the other corner formed by distal circular marker 122 with second contour line L2. As shown in FIG. 3D, system 10 is configured to illustrate a graphic representation 29 of penetration path graph 132 over the digital image on the screen.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’.

The term ‘about’, as used herein, refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

All publications, patents, and or/and patent applications, cited or referred to in this disclosure are herein incorporated in their entirety by reference into the specification, to the same extent as if each individual publication, patent, or/and patent application, was specifically and individually indicated to be incorporated herein by reference. In addition, citation or identification of any reference in this specification shall not be construed or understood as an admission that such reference represents or corresponds to prior art of the present invention. To the extent that section headings are used, they should not be construed as necessarily limiting.

What is claimed is:

1. A surgical device, comprising:
an elongated body comprising a longitudinal axis, configured to pass through an opening in a subject's body and to reach, with a distal end thereof, a surface of an organ within the subject's body;
a tissue penetrating apparatus configured to extend from the elongated body distal end through tissue layers of the organ, along a curved penetration path restricted to a chosen penetration plane;
a light projector connected to the elongated body proximally to the elongated body distal end, configured to generate a shaped illumination on the surface of the organ indicative of an intersection of the penetration plane with the surface of the organ; and
visible markers provided on the elongated body indicative of a spatial orientation of the curved penetration path over the penetration plane, relative to a visual line of sight directed generally towards the visible markers.

2. The surgical device according to claim 1, wherein the visible markers include a distal circular marker and a proximal circular marker surrounding the cylindrical portion perpendicularly to the longitudinal axis.

3. The surgical device according to claim 2, wherein the visible markers include a discrete marker.

4. The surgical device according to claim 3, wherein the discrete marker is provided between the distal circular marker and a proximal circular marker.

5. A system for processing a digital image of a portion of the surgical device of claim 3 in the subject's body relative to the visual line of sight, wherein the system comprises at least one processor, and wherein the at least one processor is configured to:
locate corners formed by intersections of the distal circular marker, the proximal circular marker and at least one contour line of the elongated body, for determining orientation of the longitudinal axis;
calculate relative positions and/or distances between the discrete marker and the corners, for determining orientation of the penetration plane relative to the longitudinal axis; and
extrapolate the spatial orientation of the curved penetration path over the penetration plane.

6. The system according to claim 5, wherein the at least one processor is configured to produce a penetration path graph for predicting the penetration path location and orientation in the organ based on the extrapolated spatial orientation and predetermined shape and size values of the tissue penetration apparatus when fully extended from the distal end of the elongated body.

7. The system according to claim 6, being connectable to a screen and configured to illustrate a graphic representation of the penetration path graph over the digital image on the screen.

8. A method comprising:
positioning a surgical device according to claim 1 in a subject's body such that the distal end of the elongated body engages with a surface of the organ;
recording a digital image capturing the surface of the organ and the visible markers from the visual line of sight;
processing the image to determine the spatial orientation of the curved penetration path over the penetration plane, relative to the visual line of sight;
producing a penetration path graph of a predicted penetration path location and orientation in the organ based on the extrapolated spatial orientation and predetermined shape and size values of the tissue penetration apparatus when fully extended from the distal end of the elongated body;
illustrating on a screen a graphic representation of the penetration path graph over the digital image.

9. The method according to claim 8, wherein the visible markers include and a distal circular marker and a proximal circular marker surrounding the cylindrical portion perpendicularly to the longitudinal axis, and a discrete marker provided in proximity to and/or between the distal circular marker and a proximal circular marker; the processing includes:
locating corners formed by intersections of the distal circular marker, the proximal circular market and at least one contour line of the elongated body, for determining orientation of the longitudinal axis;
calculating relative positions and/or distances between the discrete marker and the corners, for determining orientation of the penetration plane relative to the longitudinal axis; and
extrapolating the spatial orientation of the curved penetration path over the penetration plane.

10. The method according to claim 8, further comprising predicting an exit point of the tissue penetrating apparatus from the organ by identifying an intersection of the graphic representation with a shaped illumination on the surface of the organ, shown in the digital image, indicative of an intersection of the penetration plane with the surface of the organ.

11. The method according to claim 8, comprising projecting the laser line-shaped beam to generate the shaped illumination on the surface of the organ.

12. A system for predicting a curved penetration path of a surgical device,
the surgical device comprising:
an elongated body comprising a longitudinal axis, configured to pass through an opening in a subject's body and to reach, with a distal end thereof, a surface of an organ within the subject's body;
a tissue penetrating apparatus configured to extend from the elongated body distal end through tissue layers of the organ, along a curved penetration path restricted to a chosen penetration plane; and
visible markers provided on the elongated body indicative of a spatial orientation of the curved penetration path over the penetration plane, relative to a visual line of sight directed generally towards the visible markers, wherein the visible markers include a discrete marker, a distal circular marker and a proximal circular marker surrounding the cylindrical portion perpendicularly to the longitudinal axis;
the system comprising at least one processor for processing a digital image capturing a portion of the elongated body in the subject's body relative to the line of sight;
wherein the at least one processor is configured to:
locate corners formed by intersections of the distal circular marker, the proximal circular market and at least one contour line of the elongated body, for determining orientation of the longitudinal axis;
calculate relative positions and/or distances between the discrete marker and the corners, for determining orientation of the penetration plane relative to the longitudinal axis; and
extrapolate the spatial orientation of the curved penetration path over the penetration plane.

13. The system according to claim 12, wherein the at least one processor is configured to produce a penetration path graph for predicting the penetration path location and orientation in the organ based on the extrapolated spatial orientation and predetermined shape and size values of the tissue penetration apparatus when fully extended from the distal end of the elongated body.

14. The system according to claim 13, being connectable to a screen and configured to illustrate a graphic representation of the penetration path graph over the digital image on the screen.
